(19) United States
(12) Patent Application Publication
(10) Pub. No.: US 2006/0040864 A1
(43) Pub. Date: Feb. 23, 2006

(54) Apparatus and method for transdermal delivery of vascular endothelial growth factors

(76) Inventors: Mahmoud Ameri, Fremont, CA (US); Michel J.N. Cormier, Mountain View, CA (US); Yuh-Fun Maa, Millbrae, CA (US); Peter Daddona, Menlo Park, CA (US); Frank Stonebanks, Wayne, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/206,698

(22) Filed: Aug. 17, 2005

Related U.S. Application Data
(60) Provisional application No. 60/603,071, filed on Aug. 19, 2004.

(51) Int. Cl. A61K 38/18; A61M 31/00

(52) U.S. Cl. 514/12; 604/500

(57) ABSTRACT

An apparatus and method for transdermally delivering a biologically active agent comprising a delivery system having a microprojection member (or assembly) that includes a plurality of microprojections (or array thereof) that are adapted to pierce through the stratum corneum into the underlying epidermis layer, or epidermis and dermis layers. In one embodiment, the VEGF-based agent is contained in a biocompatible coating that is applied to the microprojection member.

### CROSS-REFERENCE TO RELATED APPLICATIONS

This application claims the benefit of U.S. Provisional Application No. 60/603,071, filed Aug. 19, 2004.

### FIELD OF THE PRESENT INVENTION

The present invention relates generally to transdermal agent delivery systems and methods. More particularly, the invention relates to an apparatus and method for transdermal delivery of vascular endothelial growth factor (VEGF)-based agents.

### BACKGROUND OF THE INVENTION

It is well known that pre-eclampsia is a syndrome of hypertension, edema and proteinuria. Pre-eclampsia affects 5 to 10% of pregnancies and results in substantial fetal morbidity and mortality. Pre-eclampsia also accounts for at least 200,000 maternal deaths worldwide per year.

Pre-eclampsia can vary in severity from mild to life threatening. A mild form of pre-eclampsia can be treated with bed rest and frequent monitoring. For moderate to severe cases, hospitalization is recommended and blood pressure medication or anticonvulsant medications to prevent seizures are prescribed. If the condition becomes life threatening to the mother or the baby, the pregnancy is typically terminated and the baby is delivered pre-term.

The proper development of the fetus and the placenta is mediated by several growth factors. One of these growth factors is the vascular endothelial growth factor (VEGF). VEGF is an endothelial cell-specific mitogen, an angiogenic inducer and a mediator of vascular permeability.

VEGF has also been shown to be important for glomerular capillary repair. VEGF binds as a homodimer to one of two homologous membrane-spanning tyrosine kinase receptors, the fins-like tyrosine kinase (Flt-1) and the inase domain eceptor (KDR), which are differentially expressed in endothelial cells obtained from many different tissues. Flt-1 (but not KDR) is highly expressed by trophoblast cells that contribute to placental formation.

Placental growth factor (P1GF) is a VEGF family member that is also involved in placental development. P1GF is expressed by cytotrophoblasts and syncytiotrophoblasts and is capable of inducing proliferation, migration and activation of endothelial cells. P1GF binds as a homodimer to the Flt-1 receptor, but not the KDR receptor. Both P1GF and VEGF contribute to the mitogenic activity and angiogenesis that are critical for the developing placenta.

It has been recently been found that levels of sFlt-1 (a splice variant of the Flt-1 receptor) are markedly elevated in placental tissue samples obtained from pregnant women suffering from pre-eclampsia. sFlt-1 is known to antagonize VEGF and PIGF by acting as a “physiologic sink” and, in pre-eclampsia or eclampsia women, sFlt-1 may cause a depletion of the necessary amounts of these essential angiogenic and miogenic factors in the placenta. It has further been opined that excess sFlt-1 may also lead to eclampsia by disrupting the endothial cells that maintain the blood-brain barrier and/or endothelial cells living the choroids plexus of the brain, thus leading to cerebral edema and seizures that are often experienced by eclamptic women. See, e.g., PCT Pub. No. WO 2004/008946.

Various VEGF-based agents and compounds have thus been administered to pre-eclamptic or eclamptic women to increase VEGF and PIGF levels to control the effects of elevated sFlt-1. Illustrative are the VEGF-based agents and compounds disclosed in PCT Pub. No. WO 2004/008946 and U.S. patent Pub. No. 2002/0137680, which are incorporated by reference herein.

Despite the efficacy of VEGF-based agents in treating disorders, such as pre-eclampsia, there are several drawbacks and disadvantages associated with the disclosed prior art methods of delivering VEGF-based agents particularly, via subcutaneous injection. A major drawback is that subcutaneous injection is a difficult, painful and uncomfortable procedure, which often results in poor patient compliance.

Transdermal delivery thus provides for an effective, alternative method of administering VEGF-based agents and compounds that would otherwise need to be delivered via hypodermic injection or intravenous infusion. The word “transdermal”, as used herein, is generic term that refers to delivery of an active or therapeutic agent through the skin to the local tissue or systemic circulatory system without substantial cutting or penetration of the skin, such as cutting with a surgical knife or piercing the skin with a hypodermic needle. Transdermal agent delivery thus includes intracutaneous, intradermal and intraepidermal delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and devices developed to mechanically penetrate or disrupt the outermost skin layers thereby creating pathways into the skin in order to enhance the amount of agent being transdermally delivered. Illustrative is the drug delivery device disclosed in U.S. Pat. No. 3,964,482, which is incorporated by reference herein.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The disclosed piercing elements generally extend perpendicularly from a thin, flat member, such as a pad or sheet. The piercing elements devices are typically extremely small; having a microprojection length of only about 25-400 microns and a microprojection thickness of only about 5-50 microns.

The disclosed systems further typically include a reservoir for holding the agent and a delivery system to transfer the agent from the reservoir through the stratum corneum, such as by hollow needles or tines. One example of such a device is disclosed in PCT Pub. No. WO 93/17754, which has a liquid agent reservoir.

As disclosed in U.S. patent application Ser. No. 10/045,842, which is fully incorporated by reference herein, it is possible to have the active agent that is to be delivered to a subject or patient coated on the microprojections instead of contained in a physical reservoir. This eliminates the necessity of a separate physical reservoir and developing an agent formulation or composition specifically for the reservoir.

It is therefore an object of the present invention to provide a transdermal agent delivery apparatus and method that facilitates minimally invasive administration of VEGF-based agents to a subject.

It is therefore an object of the present invention to provide a transdermal agent delivery apparatus and method that provides intracutaneous delivery of VEGF to a subject.

It is another object of the invention to provide a transdermal agent delivery apparatus and method for treating or preventing pre-eclampsia or eclampsia.

It is another object of the invention to provide a VEGF-based formulation for intracutaneous delivery to a subject.

It is another object of the present invention to provide a transdermal agent delivery apparatus and method that includes microprojections coated with a biocompatible coating that includes a VEGF-based formulation.

Another object of the present invention is to provide a transdermal agent delivery apparatus and method that includes a gel pack adapted to receive a hydrogel formulation that contains a VEGF-based formulation.

It is yet another object of the present invention to provide a transdermal agent delivery apparatus and method that includes a solid state form of a VEGF-based formulation that is adapted to be reconstituted prior to delivery by a hydrogel.

### SUMMARY OF THE INVENTION

In accordance with the above objects and those that will be mentioned and will become apparent below, the apparatus and method for transdermally delivering a VEGF-based agent to a subject in accordance with this invention generally comprises a delivery system having a microprojection member (or assembly) that includes a plurality of microprojections (or array thereof) that are adapted to pierce through the stratum corneum into the underlying epidermis layer, or epidermis and dermis layers. In a preferred embodiment, the microprojection member includes a biocompatible coating having at least one VEGF-based agent disposed therein. In another embodiment, the microprojection member includes a hydrogel formulation having at least one VEGF-based agent. In yet another embodiment, the microprojection member includes a solid state formulation having at least one VEGF-based agent and a hydrating hydrogel formulation.

In one embodiment of the invention, the microprojection member has a microprojection density of at least approximately 10 microprojections/cm², more preferably, in the range of at least approximately 200-2000 microprojections/cm².

In one embodiment, the microprojection member is constructed out of stainless steel, titanium, nickel titanium alloys, or similar biocompatible materials.

In another embodiment, the microprojection member is constructed out of a non-conductive material, such as a polymeric material. Alternatively, the microprojection member can be coated with a non-conductive material, such as Parylene®, or a hydrophobic material, such as Teflon®, silicon or other low energy material.

The coating formulations applied to the microprojection member to form solid biocompatible coatings can comprise aqueous and non-aqueous formulations. Preferably, the coating formulations include at least one VEGF-based agent, which can be dissolved within a biocompatible carrier or suspended within the carrier.

Preferably, the VEGF-based agent comprises a VEGF family member, including, but not limited to, isoforms of VEGF 206, VEGF 189, VEGF 183, VEGF 165, VEGF 148, VEGF 145 and VEGF 121, and salts and simple derivatives thereof. In a preferred embodiment of the invention, the VEGF-based agent comprises VEGF 121.

Throughout this application, the terms “VEGF-based agent” and “VEGF 121 based agent” include, without limitation, recombinant VEGF 121, synthetic VEGF 121 and VEGF 121 salts. Examples of pharmaceutically acceptable VEGF 121 salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate and sulfonate.

Preferably, the VEGF-based agent is present in the coating formulation at a concentration in the range of approximately 1-30 wt. %.

The amount of VEGF-based agent contained in the solid biocompatible coating (i.e., microprojection member or product) is preferably in the range of 1-1000 μg, more preferably, in the range of 1-500 μg, even more preferably, in the range of 1-200 μg.

Preferably, the pH of the coating formulation is below approximately pH 5.5 or above pH 7.0. More preferably, the coating formulation has a pH in the range of approximately pH 2-pH 5.5 or pH 7.0-pH 11. Even more preferably, the coating formulation has a pH in the range of approximately pH 2.5-pH 5.5 or pH 7.0-pH 10.5.

In certain embodiments of the invention, the viscosity of the coating formulation that is employed to coat the microprojections is enhanced by adding low volatility counterions. In one embodiment, wherein the pH of the coating formulation is less than pH 5.5, the VEGF-based agent has a positive charge and the viscosity-enhancing counterion comprises an acid. Preferably, the acidic counterion comprises a non-volatile weak acid having at least one acidic pKa and a melting point higher than approximately 50° C. or a boiling point higher than approximately 170° C. at Pₐₜₘ. Suitable acids include citric acid, succinic acid, glycolic acid, gluconic acid, glucuronic acid, lactic acid, malic acid, pyruvic acid, tartaric acid, tartronic acid and fumaric acid.

In another embodiment of the invention, the acidic counterion comprises a strong acid that exhibits at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another embodiment of the invention is directed to a mixture of counterions, wherein at least one counterion comprises a weak acid and at least one counterion comprises a non-volatile weak acid.

A further embodiment is directed to a mixture of counterions, wherein at least one of the counterion comprises a strong acid and at least one of the counterion comprises a weak acid having a high volatility and exhibiting at least one pKa higher than about 2 and a melting point lower than about 50° C. or a boiling point lower than about 170° C. at Pₐₜₘ. Examples of such acids include acetic acid, propionic acid, pentanoic acid and the like.

The acidic counterion is preferably present in an amount that is sufficient to neutralize the positive charge present on the VEGF-based agent at the pH of the formulation. In an additional embodiment, an excess counterion (as the free acid or as a salt) is added to control pH and to provide adequate buffering capacity.

In another embodiment of the invention, wherein the pH of the coating formulation is greater than pH 7.0, the VEGF-based agent has a negative charge and the viscosity-enhancing counterion comprises a base. In a preferred embodiment of the invention, the basic counterion comprises a weak base with low volatility having at least one acidic pKa and a melting point higher than approximately 50° C. or a boiling point higher than approximately 170° C. at Pₐₜₘ. Suitable bases include monoethanolomine, diethanolamine, triethanolamine, tromethamine, methylglucamine and glucosamine.

In another embodiment, the counterion comprises a strong base exhibiting at least one pKa greater than approximately 12. Suitable strong bases include sodium hydroxide, potassium hydroxide, calcium hydroxide and magnesium hydroxide.

Another embodiment of the invention is directed to a mixture of counterions wherein at least one of the counterions comprises a strong base and at least one of the counterions comprises a weak base with low volatility.

A further embodiment is directed to a mixture of counterions, wherein at least one of the counterion comprises a strong base and at least one of the counterion comprises a weak base having a high volatility and exhibiting at least one pKa lower than about 12 and a melting point lower than about 50° C. or a boiling point lower than about 170° C. at Pₐₜₘ. Examples of such bases include ammonia and morpholine.

In the noted embodiments of the invention, the basic counterion is preferably sufficient to neutralize the negative charge of the VEGF-based agent at the pH of the formulation. In additional embodiments, excess counterion (as the free acid or as a salt) is added to control pH and provide adequate buffering capacity.

In another embodiment of the invention, the coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine, monoethanolamine, diethanolamine, triethanolamine, tromethamine, lysine, histidine, arginine, morpholine, methylglucamine, glucosamine, and mixtures thereof.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan lauratealkoxylated alcohols, such as laureth-4 and polyoxyethylene castor oil derivatives, such as Cremophor EL®.

In the noted embodiments of the invention, the concentration of the surfactant is preferably in the range of approximately 0.001-2 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hydroxyethyl starch, carboxymethyl cellulose and salts of, dextran, poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 0.1-20 wt. %, more preferably, in the range of approximately 0.03-10 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and insulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose and the like.

Preferably, the concentration of the stabilizing agent in the coating formulation is at a ratio of approximately 0.1-2.0:1 with respect to the VEGF-based agent.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

Preferably, the coating formulations have a viscosity less than approximately 500 centipoise and greater than 3 centipoise.

In one embodiment of the invention, the thickness of the biocompatible coating is less than 25 microns, more preferably, less than 10 microns, as measured from the microprojection surface.

In a further embodiment of the invention, the delivery system includes a hydrogel formulation. Preferably, the hydrogel formulation is contained in a gel pack.

In at least one embodiment of the invention, the hydrogel formulation contains at least one VEGF-based agent.

Preferably, the VEGF-based agent is present at a concentration in excess or below saturation.

In one embodiment of the invention, the VEGF-based agent comprises in the range of approximately 1-40 wt. % of the hydrogel formulation.

Preferably, the hydrogel formulations comprise water-based hydrogels having macromolecular polymeric networks.

In a preferred embodiment of the invention, the polymeric network comprises, without limitation, hyroxyethyl starch, dextran, hydroxyethylcellulose (HEC), hydroxypropylmethylcellulose (HPMC), hydroxypropycellulose (HPC), methylcellulose (MC), hydroxyethyl-methylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone) and pluronics.

The hydrogel formulation preferably includes at least one surfactant, which can be zwitterionic, amphoteric, cationic, anionic or nonionic.

In one embodiment of the invention, the surfactant comprises sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols, such as laureth-4.

In another embodiment, the hydrogel formulation includes polymeric materials or polymers having amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC) and ethylhydroxyethylcellulose (EHEC), as well as pluronics.

In a further embodiment of the invention, the hydrogel formulation contains at least one pathway patency modulator, which can comprise, without limitation, osmotic agents (e.g., sodium chloride), zwitterionic compounds (e.g., amino acids), and anti-inflammatory agents, such as betamethoasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinate sodium salt, paramethasone disodium phosphate and prednisolone 21-succinate sodium salt, and anticoagulants, such as citric acid, citrate salts (e.g., sodium citrate), dextrin sulfate sodium and EDTA.

In yet another embodiment of the invention, the hydrogel formulation includes at least one vasoconstrictor, which can comprise, without limitation, epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropolamine, propylhexedrine, pseudoephedrine, tetrahydrozoline, tramazoline, tuaminoheptane, tymazoline, vasopressin and xylometazoline, and the mixtures thereof.

In accordance with yet another embodiment of the invention, the delivery system for delivering a VEGF-based agent includes a microprojection member having top and bottom surfaces, a plurality of openings that extend through the microprojection member and a plurality of microprojections that project from the bottom surface of the microprojection member. The microprojection member further includes a hydrogel formulation and a solid state formulation having at least one VEGF-based agent, preferably, VEGF 121.

In one embodiment, the solid state formulation is disposed proximate the top surface of the microprojection member. In another embodiment, the solid state formulation is disposed proximate the bottom surface of the microprojection member.

In one embodiment of the invention, the hydrogel formulation is devoid of a VEGF-based agent and, hence, is merely a hydration mechanism.

In one embodiment of the invention, the solid state formulation is a solid film. Preferably, the solid film is made by casting a liquid formulation consisting of at least one VEGF-based agent, a polymeric material, such as hyroxyethyl starch, dextran, hydroxyethylcellulose (HEC), hydroxypropylmethylcellulose (HPMC), hydroxypropycellulose (HPC), methylcellulose (MC), hydroxyethylmethylcellulose (HEMC), ethylhydroxethylcellulose (EHEC), carboxymethylcellulose (CMC), poly(vinyl alcohol), poly(ethylene oxide), poly(2-hydroxyethymethacrylate), poly(n-vinyl pyrolidone) and pluronics, a plasticising agent, such as glycerol, propylene glycol, and polyethylene glycol, a surfactant, such as Tween 20 and Tween 80, and a volatile solvent, such as water, isopropanol, methanol and ethanol.

In other embodiments of the invention, the solid state formulation is formed by a process selected from the group consisting of spray drying, freeze drying, spray freeze drying and supercritical fluid extraction. A currently preferred process is spray freeze drying. In the noted embodiments, the biocompatible coating is adapted to be reconsitituted by a suitable solvent in up to approximately 15 min, and preferably, in up to approximately 1 min. The coating formulation also preferably includes an antioxidant.

In accordance with one embodiment of the invention, the method for delivering a VEGF-based agent to a patient includes the following steps: (i) providing a delivery system having a microprojection member, the microprojection member including a plurality of microprojections and a biocompatible coating having at least one VEGF-based agent, (ii) applying the coated microprojection member to the patient's skin, whereby the microprojections pierce the skin and the agent-containing coating is dissolved by body fluid and released into the skin.

The coated microprojection member is preferably left on the patient's skin for a period lasting from 5 seconds to 24 hours. Following the desired wearing time, the microprojection member is removed from the skin.

In accordance with a further embodiment of the invention, the method for delivering a VEGF-based agent to a patient includes the following steps: (i) providing a delivery system having a microprojection member and a gel pack including a hydrogel formulation having at least one VEGF-based agent, (ii) applying the microprojection member-gel pack assembly to the patient's skin, whereby the microprojections pierce the stratum corneum and form a plurality of microslits in the stratum corneum, and whereby the agent-containing hydrogel formulation migrates into and through the microslits formed by the microprojections.

The microprojection member-gel pack assembly is preferably left on the patient's skin for a period lasting from 5 minutes to 24 hours. Following the desired wearing time, the microprojection member-gel pack assembly is removed from the skin.

In a further aspect of the noted embodiment, the microprojection member includes an agent-containing biocompatible coating.

Preferably, the coated microprojection member-gel pack assembly (including the agent-containing hydrogel formulation) is left on the patient's skin for a period lasting from 5 seconds to 24 hours.

In a further aspect of the noted embodiment, the microprojection member includes an agent-containing biocompatible coating and the hydrogel formulation is devoid of a VEGF-based agent and, hence, is merely a hydration mechanism.

Preferably, the coated microprojection member-gel pack assembly (including the agent-containing hydrogel formulation) is left on the patient's skin for a period lasting from 5 minutes to 24 hours.

In accordance with a further embodiment of the invention, the method for delivering a VEGF-based agent to a patient includes the following steps: (i) providing a delivery system having a microprojection member and a gel pack including a hydrogel formulation having at least one VEGF-based agent, (ii) applying the microprojection member to the patient's skin, whereby the microprojections pierce the stratum corneum and form a plurality of microslits in the stratum corneum, and (iii) placing the gel pack on top of the applied microprojection member, whereby the agent-containing hydrogel formulation migrates into and through the microslits formed by the microprojections.

The microprojection member-gel pack assembly is preferably left on the patient's skin for a period lasting from 5 minutes to 24 hours. Following the desired wearing time, the microprojection member-gel pack assembly is removed from the skin.

In a further aspect of the noted embodiment, the microprojection member includes an agent-containing biocompatible coating and the hydrogel formulation is devoid of a VEGF-based agent and, hence, is merely a hydration mechanism.

In accordance with another embodiment of the invention, the method for delivering a VEGF-based agent includes the following steps: (i) providing a delivery system having a microprojection member and a gel pack including a hydrogel formulation having at least one VEGF-based agent, (ii) applying the microprojection member to the patient's skin, whereby the microprojections pierce the stratum corneum and form a plurality of microslits in the stratum corneum, (iii) removing the microprojection member from the patient's skin, and (iv) placing the gel pack on top of the pretreated skin, whereby the agent-containing hydrogel formulation migrates into and through the microslits formed by the microprojections.

The gel pack is preferably left on the patient's skin for a period lasting from 5 minutes to 24 hours. Following the desired wearing time, the gel pack is removed from the skin.

In yet another embodiment of the invention, the microprojection member having a VEGF-based agent containing biocompatible coating is applied to the patient's skin, a gel pack having a VEGF-based agent containing hydrogel formulation is then placed on top of the applied microprojection member, whereby the agent-containing coating is dissolved by body fluid and released into the skin and the agent-containing hydrogel formulation migrates into and through the microslits in the stratum corneum formed by the microprojections.

The microprojection member-gel pack assembly is preferably left on the patient's skin for a period lasting 5 minutes to 24 hours. Following the desired wearing time, the microprojection member and gel pack are removed.

In a further embodiment of the invention, the method for delivering a VEGF-based agent includes the following steps: (i) providing a microprojection assembly having a microprojection member, a hydrogel formulation and a solid state formulation having at least one VEGF-based agent, and (ii) applying the microprojection assembly to the patient's skin, whereby the microprojections pierce the stratum corneum, the hydrogel formulation hydrates and releases the agent from the solid state formulation and the agent migrates into and through the microslits in the stratum corneum formed by the microprojections.

The microprojection member is preferably left on the patient's skin for a period lasting from 5 minutes to 24 hours. Following the desired wearing time, the microprojection member is removed from the skin.

In a further embodiment of the invention, the method for delivering a VEGF-based agent includes the following steps: (i) providing a microprojection assembly having a microprojection member and a solid state formulation having at least one VEGF-based agent, (ii) providing a gel pack having a hydrogel formulation, (iii) applying the microprojection assembly to the patient's skin, whereby the microprojections pierce the stratum corneum, and (iv) placing the gel pack on the applied microprojection assembly, whereby the hydrogel formulation is released from the gel pack and releases the agent contained in the solid state formulation and the agent and hydrogel formulation migrate into and through the microslits in the stratum corneum formed by the microprojections.

The microprojection member is preferably left on the patient's skin for a period lasting from 5 minutes to 24 hours. Following the desired wearing time, the microprojection member is removed from the skin.

Preferably, the microprojection members (and assemblies) and microprojection member-gel pack assemblies employed herein are applied to the patient's skin via an actuator.

Preferably, the dose of VEGF-based agent delivered intracutaneously via the aforementioned methods is in the range of approximately 1-500 μg, more preferably, in the range of approximately 1-100 μg, even more preferably, in the range of approximately 1-50 μg per dosage unit.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection member;

FIG. 2 is a perspective view of the microprojection member shown in FIG. 1 having a coating deposited on the microprojections, according to the invention;

FIG. 3 is a side sectional view of a microprojection member having an adhesive backing;

FIG. 4 is a side sectional view of a retainer having a microprojection member disposed therein;

FIG. 5 is a perspective view of the retainer shown in FIG. 4;

FIG. 6 is an exploded perspective view of an applicator and retainer;

FIG. 7 is a graph illustrating the charge profile for a VEGF-based agent; and

FIGS. 8 and 9 are graphs illustrating the mole ratios of a net-charged species of a VEGF-based agent.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a VEGF-based agent” includes two or more such agents; reference to “a microprojection” includes two or more such microprojections and the like.

### DEFINITIONS

The term “transdermal”, as used herein, means the delivery of an agent into and/or through the skin for local or systemic therapy. The term “transdermal” thus means and includes intracutaneous, intradermal and intraepidermal delivery of an agent, such as a peptide, into and/or through the skin via passive diffusion as well as energy-based diffusional delivery, such as iontophoresis and phonophoresis.

The term “transdermal flux”, as used herein, means the rate of transdermal delivery.

The term “subject”, as used herein, means a mammal, including, but not limited to, a human or non-human mammal, such as a bovine, equine, canine, ovine or feline. The term “subject” further includes pregnant, post-partum and non-pregnant mammals.

The term “pre-eclampsia”, as used herein, means a multi-system disorder that is characterized by hypertension with proteinuria or edema or both, glomerular dysfunction, cerebral edema, pulmonary edema, liver edema or coagulation abnormalities due to pregnancy or the influence of the recent pregnancy. Pre-eclampsia generally occurs after the 20th week of gestation and is often reflected by a combination of the following symptoms: (1) a systolic blood pressure (BP) 140 mmHg and a diastolic BP 90 mmHg after 20 weeks gestation, (2) new onset proteinuria (1+by dipstick on urinanaysis, >300 mg of protein in a 24-hour urine collection, or a single random urine sample having a protein/creatinine ratio >0.3) and (3) resolution of hypertension and proteinuria by 12 weeks postpartum. The symptoms of pre-eclampsia can also include renal dysfunction and glomerular endotheliosis or hypertrophy.

In pre-eclampsia, hypertension and proteinuria generally occur within seven days of each other. In severe pre-eclampsia, severe hypertension, severe proteinuria and HELLP syndrome (hemolysis, elevated liver enzymes, low platelets) or eclampsia can occur simultaneously or only one symptom at a time.

The terms “symptoms of pre-eclampsia” and “symptoms of eclampsia”, as used herein, mean the development of any of the aforementioned symptoms due to pregnancy or the influence of recent pregnancy, including seizures and coma.

The term “treat”, as used herein, means administering a therapeutic agent or a pharmaceutical composition for prophylactic and/or therapeutic purposes.

The term “therapeutic treatment”, as used herein, refers to administering treatment to a subject already suffering from a disease to improve the subject's condition. Preferably, the subject is diagnosed as suffering from pre-eclampsia or eclampsia based on identification of any of the characteristic symptoms described herein.

To term “preventative treatment”, as used herein, refers to prophylactic treatment of a subject who is not yet ill, but who is susceptible to, or otherwise at risk of, developing a particular disease.

The terms “vascular endothelial growth factor” and “VEGF”, as used herein, mean a mammalian growth factor that is homologous to the growth factors defined in U.S. Pat. Nos. 5,332,671, 5,240,848, 5,194,596 and 5,219,739, which are expressly incorporated by reference herein, and has VEGF biological activity. The biological activity of native VEGF includes the promotion of selective growth of vascular endothelial cells or umbilical vein endothelial cells and induction of angiogenesis.

The terms “VEGF-based agent” and “VEGF 121 based agent”, as used herein, include, without limitation, all VEGF family members, including, but not limited to, isoforms of VEGF 206, VEGF 189, VEGF 183, VEGF 165, VEGF 148, VEGF 145, VEGF 121, bVEGF 120, bVEGF 164, hVEGF 121 and hVEGF 165, and salts, variants, analogs, simple derivatives and combinations thereof.

The terms “VEGF-based agent” and “VEGF 121 based agent” thus include, without limitation, recombinant VEGF 121, synthetic VEGF 121 and VEGF 121 salts. Examples of pharmaceutically acceptable VEGF 121 salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate and sulfonate.

It is to be understood that more than one VEGF-based agent can be incorporated into the agent source, reservoirs, and/or coatings of this invention, and that the use of the term “VEGF-based agent” in no way excludes the use of two or more such agents.

The term “biologically effective amount” or “therapeutically effective amount”, as used herein, means the amount or rate of the VEGF-based agent needed to effect the desired therapeutic, often beneficial, result. The amount of VEGF-based agent employed in the coatings of the invention will thus be that amount necessary to deliver a therapeutically effective amount of the VEGF-based agent to achieve the desired therapeutic result. In practice, this will vary depending upon the particular VEGF-based agent being delivered, the site of delivery, the severity of the condition being treated, the desired the desired therapeutic effect and the dissolution and release kinetics for delivery of the VEGF-based agent from the coating into skin tissues.

Preferably, the therapeutically effective amount of the VEGF-based agent administered to a subject suffering from pre-eclampsia or eclampsia is sufficient to cause a qualitative or quantitative reduction in the symptoms of pre-eclampsia or eclampsia, as described herein.

The term “co-delivering”, as used herein, means that a supplemental agent(s) is administered transdermally either before the VEGF-based agent is delivered, before and during transdermal flux of the VEGF-based agent, during transdermal flux of the VEGF-based agent, during and after transdermal flux of the VEGF-based agent, and/or after transdermal flux of the VEGF-based agent. Additionally, two or more VEGF-based agents may be formulated in the coatings and/or formulations, resulting in co-delivery of the VEGF-based agents.

The terms “microprojections” and “microprotrusions”, as used herein, refers to piercing elements that are adapted to pierce or cut through the stratum corneum into the underlying epidermis layer, or epidermis and dermis layers, of the skin of a subject, particularly a living mammal, more particularly, a human.

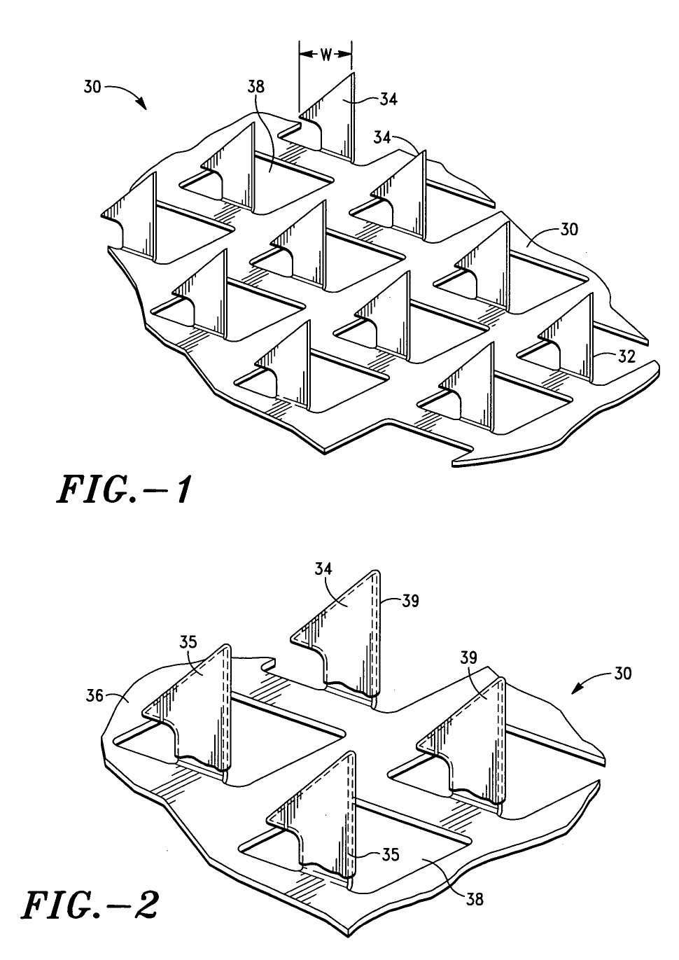

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 1. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture that is employed to coat the microprojections and/or arrays thereof. Preferably, the coating formulation includes at least one VEGF-based agent, which can be in solution or suspension in the formulation.

The term “biocompatible coating” and “solid coating”, as used herein, is meant to mean and include a “coating formulation” in a substantially solid state.

The term “solid state formulation”, as used herein, is meant to mean and include solid films formed by casting, and powders or cakes formed by spray drying, freeze drying, spray freeze drying and supercritical fluid extraction.

As indicated above, the present invention generally comprises a delivery system including microprojection member (or system) having a plurality of microprojections (or array thereof) that are adapted to pierce through the stratum corneum into the underlying epidermis layer, or epidermis and dermis layers. The microprojection member (or system) includes at least one agent source or agent delivery medium (i.e., biocompatible coating, hydrogel formulation, or solid state formulation). In a preferred embodiment, the microprojection member includes a biocompatible coating having at least one VEGF-based agent disposed thereon.

As stated, “pre-eclampsia”, is a multi-system disorder that is typically characterized by hypertension with proteinuria or edema or both, glomerular dysfunction, cerebral edema, liver edema, pulmonary edema or coagulation abnormalities, due to pregnancy or the influence of the recent pregnancy. Pre-eclampsia generally occurs after the 20th week of gestation. The symptoms often associated with pre-eclampsia comprise: (1) a systolic blood pressure (BP) 140 mmHg and a diastolic BP 90 mmHg after 20 weeks gestation, (2) new onset proteinuria (1+by dipstick on urinanaysis, >300 mg of protein in a 24-hour urine collection, or a single random urine sample having a protein/creatinine ratio >0.3) and (3) resolution of hypertension and proteinuria by 12 weeks postpartum. The symptoms of pre-eclampsia can also include renal dysfunction and glomerular endotheliosis or hypertrophy.

Severe pre-eclampsia is generally defined as (1) a diastolic BP 110 mmHg or (2) proteinuria characterized by a measurement of 3.5 g or more protein in a 24-hour urine collection or two random urine specimens with at least 3+protein by dipstick.

In pre-eclampsia, hypertension and proteinuria generally occur within seven days of each other. In severe pre-eclampsia, severe hypertension, severe proteinuria and HELLP syndrome (hemolysis, elevated liver enzymes, low platelets) or eclampsia can occur simultaneously or only one symptom at a time.

As discussed in detail herein, a key advantage of the present invention is that the delivery system systemically delivers the VEGF-based agent to a subject, particularly, a human patient, whereby the VEGF-based agent produces a qualitative or quantitative reduction in at least one of the noted symptoms of pre-eclampsia and/or eclampsia.

Referring now to FIG. 1, there is shown one embodiment of a microprojection member 30 for use with the present invention. As illustrated in FIG. 1, the microprojection member 30 includes a microprojection array 32 having a plurality of microprojections 34. The microprojections 34 preferably extend at substantially a 90° angle from the sheet, which in the noted embodiment includes openings 38.

According to the invention, the sheet 36 can be incorporated into a delivery patch, including a backing 40 for the sheet 36, and can additionally include adhesive 16 for adhering the patch to the skin (see FIG. 3). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

As indicated, the microprojections 34 preferably have a projection length less than 1000 microns. In one embodiment, the microprojections 34 have a projection length of less than 500 microns, more preferably, less than 250 microns. The microprojections 34 also preferably have a width in the range of approximately 25-500 microns and thickness in the range of approximately 10-100 microns.

To enhance the biocompatibility of the microprojection member 30 (e.g., minimize bleeding and irritation following application to the skin of a subject), in one embodiment of the invention, the microprojections 34 preferably have a length less than 145 μm, more preferably, in the range of approximately 50-145 μm, even more preferably, in the range of approximately 70-140 μm. Further, the microprojection member 30 comprises an array preferably having a microprojection density greater than 100 microprojections/cm², more preferably, in the range of approximately 200-3000 microprojections/cm².

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymeric material. Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. application Ser. No. 60/484,142, which is incorporated by reference herein.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the VEGF-based agent to be administered to a host can be contained in a biocompatible coating that is disposed on the microprojection member 30 or contained in a hydrogel formulation or contained in both the biocompatible coating and the hydrogel formulation.

In a further embodiment, wherein the microprojection member includes an agent-containing solid state formulation, the VEGF-based agent can be contained in the biocompatible coating, hydrogel formulation or solid state formulation, or in all three delivery mediums.

According to the invention, at least one VEGF-based agent is contained in at least one of the aforementioned delivery mediums. The amount of the VEGF-based agent that is employed in the delivery medium and, hence, microprojection system will be that amount necessary to deliver a therapeutically effective amount of the VEGF-based agent to achieve the desired result. In practice, this will vary widely depending upon the particular VEGF-based agent, the site of delivery, the severity of the condition, and the desired therapeutic effect.

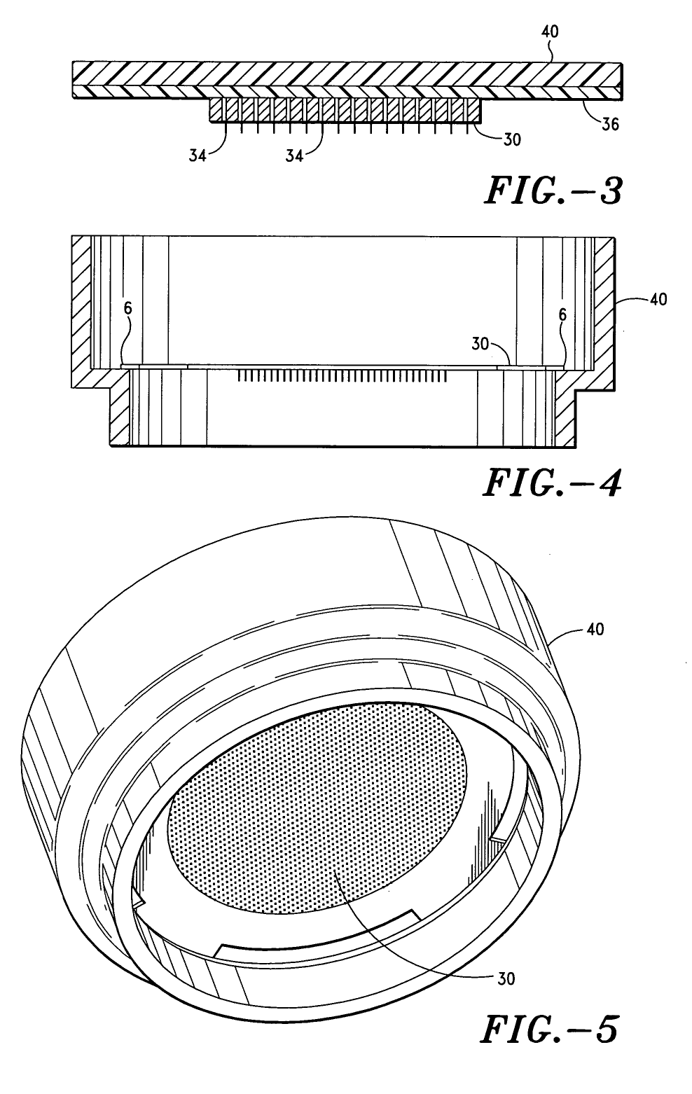

Referring now to FIG. 2, there is shown a microprojection member 30 having microprojections 34 that include a biocompatible coating 35 having a VEGF-based agent disposed therein. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No.2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

Referring now to FIGS. 4 and 5, for storage and application, the microprojection member 30 is preferably suspended in a retainer ring 40 by adhesive tabs 6, as described in detail in U.S. application Ser. No. 09/976,762 (Pub. No. 2002/0091357), which is incorporated by reference herein in its entirety.

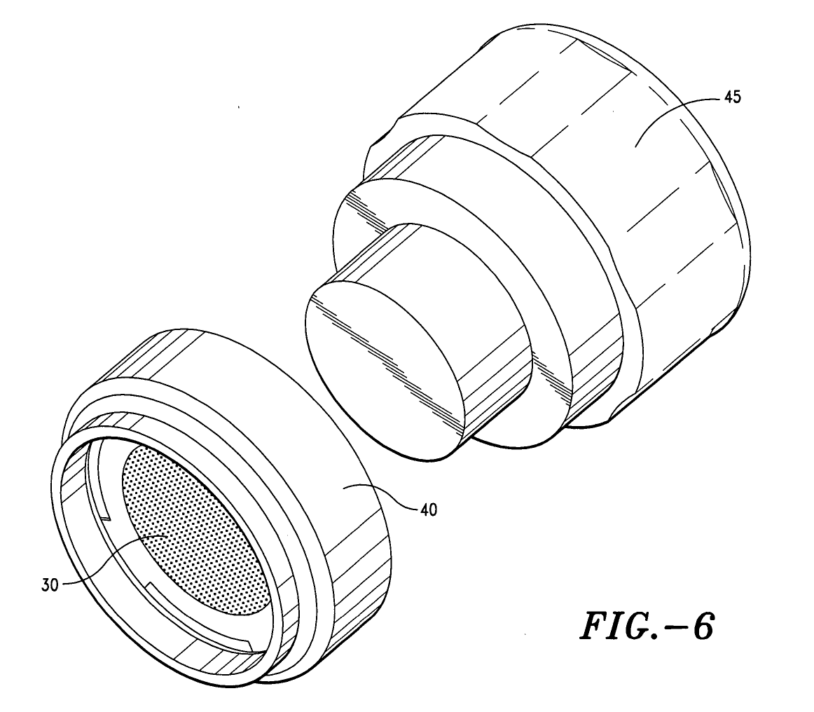

After placement of the microprojection member 30 in the retainer ring 40, the microprojection member 30 is applied to the patient's skin. Preferably, the microprojection member 30 is applied to the patient's skin using an impact applicator 45, such as shown in FIG. 6 and described in Co-Pending U.S. application Ser. No. 09/976,978, which is incorporated by reference herein in its entirety.

As indicated, according to one embodiment of the invention, the coating formulations applied to the microprojection member 30 to form solid biocompatible coatings can comprise aqueous and non-aqueous formulations having at least one VEGF-based agent. According to the invention, the VEGF-based agent can be dissolved within a biocompatible carrier or suspended within the carrier.

Referring now to FIG. 7, there is shown the predicted charge profile of VEGF 121, a protein exhibiting 25 acidic pKa's and 23 basic pKa's. As illustrated in FIG. 7, at pH 6.0, the protein presents a zero net electric charge. This point is also called the isoelectric point or pI.

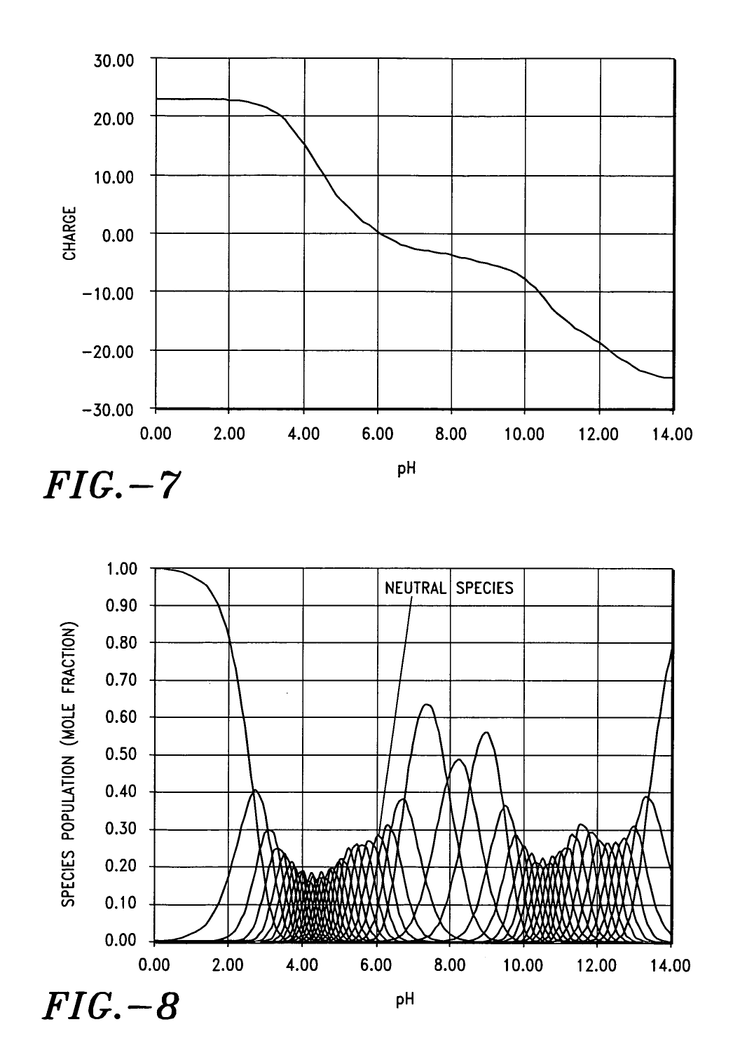

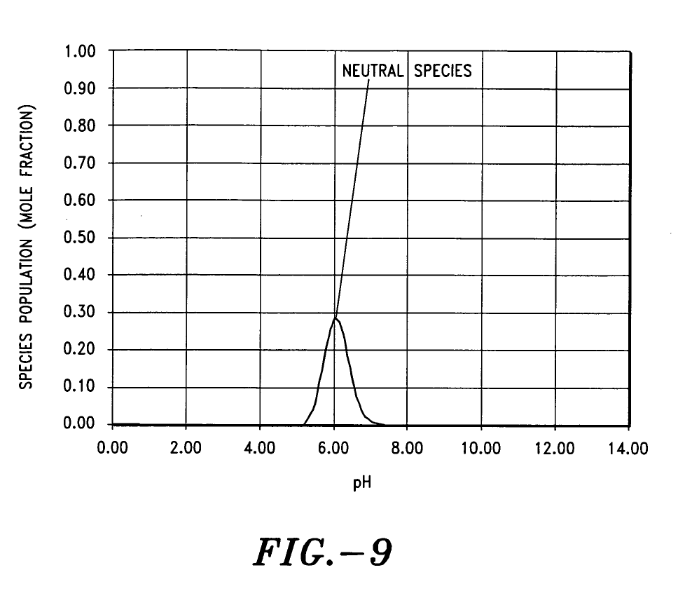

Referring now to FIG. 8, there is shown the predicted mole ratios of the net charged species of VEGF 121. As illustrated in FIG. 8, the neutral species only exist in significant amounts in the pH range of pH 5.5 to pH 7.0 (see also FIG. 9). In this pH range, the protein is expected to precipitate out of solution.

The data thus reflects that VEGF 121 solubility compatible with formulations acceptable for coating on a microprojection array of the invention can be achieved at a pH below about pH 5.5 or above pH 7.0. Accordingly, the preferred ranges of pH for the coating formulations of the invention are pH 2-pH 5.5 or pH 7.0-pH 11.

Preferably, the VEGF-based agent comprises a VEGF family member, including, but not limited to, isoforms of VEGF 206, VEGF 189, VEGF 183, VEGF 165, VEGF 148, VEGF 145 and VEGF 121, and salts, variants, analogs, and simple derivatives thereof. In a preferred embodiment of the invention, the VEGF-based agent comprises VEGF 121.

Throughout this application, the terms “VEGF-based agent” and “VEGF 121 based agent” include, without limitation, recombinant VEGF 121, synthetic VEGF 121 and VEGF 121 salts. Examples of pharmaceutically acceptable VEGF 121 salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate and sulfonate.

Preferably, the VEGF-based agent is present in the coating formulation at a concentration in the range of approximately 1-30 wt. %.

The amount of VEGF-based agent contained in the solid biocompatible coating (i.e., microprojection member or product) is preferably in the range of approximately 1-1000 μg, more preferably, in the range of approximately 1-500 μg, even more preferably, in the range of approximately 1-200 μg.

Preferably, the pH of the coating formulation is below approximately pH 5.5 or above pH 7.0. More preferably, the coating formulation has a pH in the range of approximately pH 2-pH 5.5 or pH 7.0-pH 11. Even more preferably, the coating formulation has a pH in the range of approximately pH 2.5-pH 5.5 or pH 7.0-pH 10.5.

In certain embodiments of the invention, the viscosity of the coating formulation that is employed to coat the microprojections is enhanced by adding low volatility counterions. In one embodiment, wherein the pH of the coating formulation is less than pH 5.5, the VEGF-based agent has a positive charge and the viscosity-enhancing counterion comprises an acid. Preferably, the acidic counterion comprises a non-volatile weak acid having at least one acidic pKa and a melting point higher than approximately 50° C. or a boiling point higher than approximately 170° C. at Pₐₜₘ. Suitable acids include citric acid, succinic acid, glycolic acid, gluconic acid, glucuronic acid, lactic acid, malic acid, pyruvic acid, tartaric acid, tartronic acid and fumaric acid.

In another embodiment of the invention, the acidic counterion comprises a strong acid that exhibits at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another embodiment of the invention is directed to a mixture of counterions wherein at least one counterion comprises a weak acid and at least one counterion comprises a non-volatile weak acid.

A further embodiment is directed to a mixture of counterions, wherein at least one of the counterion comprises a strong acid and at least one of the counterion comprises a weak acid having a high volatility and exhibiting at least one pKa higher than about 2 and a melting point lower than about 50° C. or a boiling point lower than about 170° C. at Pₐₜₘ. Examples of such acids include acetic acid, propionic acid, pentanoic acid and the like.

The acidic counterion is preferably present in an amount that is sufficient to neutralize the positive charge present on the VEGF-based agent at the pH of the formulation. In an additional embodiment, an excess counterion (as the free acid or as a salt) is added to control pH and to provide adequate buffering capacity.

In another embodiment of the invention, wherein the pH of the coating formulation is greater than pH 7.0, the VEGF-based agent has a negative charge and the viscosity-enhancing counterion comprises a base. In a preferred embodiment of the invention, the basic counterion comprises a weak base with low volatility having at least one acidic pKa and a melting point higher than approximately 50° C. or a boiling point higher than approximately 170° C. at Pₐₜₘ. Suitable bases include monoethanolomine, diethanolamine, triethanolamine, tromethamine, methylglucamine and glucosamine.

In another embodiment, the counterion comprises a strong base exhibiting at least one pKa greater than approximately 12. Suitable strong bases include sodium hydroxide, potassium hydroxide, calcium hydroxide and magnesium hydroxide.

Another embodiment of the invention is directed to a mixture of counterions wherein at least one of the counterions comprises a strong base and at least one of the counterions comprises a weak base with low volatility.

A further embodiment is directed to a mixture of counterions, wherein at least one of the counterion comprises a strong base and at least one of the counterion comprises a weak base having a high volatility and exhibiting at least one pKa lower than about 12 and a melting point lower than about 50° C. or a boiling point lower than about 170° C. at Pₐₜₘ. Examples of such bases include ammonia and morpholine.

In the noted embodiments of the invention, the basic counterion is preferably sufficient to neutralize the negative charge of the VEGF-based agent at the pH of the formulation. In additional embodiments, excess counterion (as the free acid or as a salt) is added to control pH and provide adequate buffering capacity.

In another embodiment of the invention, the coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine, monoethanolamine, diethanolamine, triethanolamine, tromethamine, lysine, histidine, arginine, morpholine, methylglucamine, glucosamine, and mixtures thereof.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan lauratealkoxylated alcohols, such as laureth-4 and polyoxyethylene castor oil derivatives, such as Cremophor EL®.

In the noted embodiments of the invention, the concentration of the surfactant is preferably in the range of approximately 0.001-2 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hydroxyethyl starch, carboxymethyl cellulose and salts of, dextran, poly(vinyl alcohol), poly(ethylene oxide), poly(2-hydroxyethyl-methacrylate), poly(n-vinyl pyrolidone), polyethylene glycol and mixtures thereof, and like polymers.

In a preferred embodiment, the concentration of the hydrophilic polymer in the coating formulation is in the range of approximately 0.1-20 wt. %, more preferably, in the range of approximately 0.03-10 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation.

In another embodiment, the coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and insulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose and the like.

Preferably, the concentration of the stabilizing agent in the coating formulation is at a ratio of approximately 0.1-2.0:1 with respect to the VEGF-based agent.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the coating formulations and, hence, solid biocompatible coatings of the invention is particularly useful to prevent bleeding that can occur following application of the microprojection member or array and to prolong the pharmacokinetics of the VEGF based agent through reduction of the blood flow at the application site and reduction of the absorption rate from the skin site into the system circulation.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

Preferably, the coating formulations have a viscosity less than approximately 500 centipoise and greater than 3 centipoise.

In one embodiment of the invention, the thickness of the biocompatible coating is less than 25 microns, more preferably, less than 10 microns, as measured from the microprojection surface.

The desired coating thickness is dependent upon several factors, including the required dosage and, hence, coating thickness necessary to deliver the dosage, the density of the microprojections per unit area of the sheet, the viscosity and concentration of the coating composition and the coating method chosen.

According to the invention, after a coating formulation has been applied to the microprojections 34, the coating formulation can be dried by various means. In a preferred embodiment of the invention, the coated microprojection member 30 is dried in ambient room conditions. However, various temperatures and humidity levels can be used to dry the coating formulation onto the microprojections. Additionally, the coated member can be heated, lyophilized, freeze dried or similar techniques used to remove the water from the coating.

According to the invention, the VEGF-based agent can also be delivered via a hydrogel formulation. In the noted embodiment(s), the delivery system would thus include a hydrogel formulation and means for receiving same (e.g., gel pack), such as disclosed in U.S. Pat. Nos. 6,083,196 and 6,050,988 and Co-Pending U.S. patent application Ser. Nos. 10/970,901, 10/971,430, 10/971,877 and 10/971,338; which are incorporated by reference herein in their entirety.

In at least one embodiment, the hydrogel formulation contains at least one VEGF-based agent. In an alternative embodiment of the invention, the hydrogel formulation is devoid of a VEGF-based agent and, hence, is merely a hydration mechanism.

According to the invention, when the hydrogel formulation contains one of the aforementioned VEGF-based agents, the agent can be present at a concentration in excess of saturation or below saturation. The amount of VEGF-based agent employed in the hydrogel formulations of the invention will be that amount necessary to deliver a therapeutically effective amount of the VEGF-based agent to achieve the desired result.

In one embodiment of the invention, the concentration of the VEGF-based agent is in the range of at least 1-40 wt. % of the hydrogel formulation.

According to the invention, when the hydrogel formulation is devoid of a VEGF-based agent, the VEGF-based agent can coated on the microprojection member, as described above, or contained in a solid state formulation, as described below.

Preferably, the hydrogel formulations of the invention comprise water-based hydrogels. Hydrogels are preferred formulations because of their high water content and biocompatibility.

As is well known in the art, hydrogels are macromolecular polymeric networks that are swollen in water. Examples of suitable polymeric networks include, without limitation, hydroxyethylcellulose (HEC), hydroxypropylmethylcellulose (HPMC), hydroxypropycellulose (HPC), methylcellulose (MC), hydroxyethylmethylcellulose (HEMC), ethylhydroxyethyl-cellulose (EHEC), carboxymethyl cellulose (CMC), poly(vinyl alcohol), poly(ethylene oxide), poly(2-hydroxyethylmethacrylate), poly(n-vinyl pyrolidone), and pluronics. The most preferred polymeric materials are cellulose derivatives. The noted polymers can be obtained in various grades presenting different average molecular weight and therefore exhibit different rheological properties.

Preferably, the concentration of the polymeric material is in the range of approximately 0.5-40 wt. % of the hydrogel formulation.

The hydrogel formulations of the invention preferably have sufficient surface activity to insure that the formulations exhibit adequate wetting characteristics, which are important for establishing optimum contact between the formulation and the microprojection member and skin and, optionally, the solid state formulation.

According to the invention, adequate wetting properties are achieved by incorporating at least one wetting agent, such as a surfactant or polymer having amphiphilic properties, in the hydrogel formulation. Optionally, a wetting agent can also be incorporated in the solid state formulation.

According to the invention, the surfactant can be zwitterionic, amphoteric, cationic, anionic, or nonionic. Examples of suitable surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols, such as laureth-4. Most preferred surfactants include Tween 20, Tween 80, and SDS.

Suitable polymeric materials or polymers having amphiphilic properties include, without limitation, cellulose derivatives, such as hydroxyethylcellulose (HEC), hydroxypropyl-methylcellulose (HPMC), hydroxypropycellulose (HPC), methylcellulose (MC), hydroxyethyl-methylcellulose (HEMC), or ethylhydroxyethylcellulose (EHEC), as well as pluronics.

Preferably, the concentration of the surfactant is in the range of approximately 0.001-2 wt. % of the hydrogel formulation. The concentration of the polymer that exhibits amphiphilic properties is preferably in the range of approximately 0.5-40 wt. % of the hydrogel formulation.

As will be appreciated by one having ordinary skill in the art, the noted wetting agents can be used separately or in combinations.

According to the invention, the hydrogel formulation can include at least one of the aforementioned pathway patency modulators.

The hydrogel formulation can further include at least one of the aforementioned vasoconstrictors.

The hydrogel formulations of the invention exhibit adequate viscosity so that the formulation can be contained in a gel pack, keeps its integrity during the application process, and is fluid enough so that it can flow through the microprojection member openings and into the skin pathways.

For hydrogel formulations that exhibit Newtonian properties, the viscosity of the hydrogel formulation is preferably in the range of approximately 2-30 Poises (P), as measured at 25° C. For shear-thinning hydrogel formulations, the viscosity, as measured at 25° C., is preferably in the range of 1.5-30 P or 0.5 and 10 P, at shear rates of 667/s and 2667/s, respectively. For dilatant formulations, the viscosity, as measured at 25° C., is preferably in the range of approximately 1.5-30 P, at a shear rate of 667/s.

According to the invention, the VEGF-based agent can also be included in a solid state formulation. In one embodiment, the solid state formulation is a solid film, such as disclosed in PCT Pub. No. WO 98/28037, which is similarly incorporated by reference herein in its entirety. In another embodiment, the solid state formulation is a dry powder formulation, described below. The noted solid state formulations can be adapted to be positioned on the skin side of the microprojection array, such as disclosed in Co-Pending U.S. patent application Ser. No. 10/970,901, which is hereby incorporated by reference, or the top surface of the array.

As discussed in detail in the noted Co-Pending Application, a solid state formulation comprising a solid film is typically made by casting a liquid formulation consisting of the VEGF-based agent, a polymeric material, such as hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), carboxymethyl cellulose (CMC), poly(vinyl alcohol), poly(ethylene oxide), poly(2-hydroxyethylmethacrylate), poly(n-vinyl pyrolidone), or pluronics, a plasticising agent, such as glycerol, propylene glycol, or polyethylene glycol, a surfactant, such as Tween 20 or Tween 80, and a volatile solvent, such as water, isopropanol, or ethanol. Following casting and subsequent evaporation of the solvent, a solid film is produced.

In one embodiment, the liquid formulation used to produce the solid film comprises: 0.1-20 wt. % VEGF-based agent, 5-40 wt. % polymer, 5-40 wt. % plasticizer, 0-2 wt. % surfactant, and the balance comprising volatile solvent.

As noted, in other embodiments of the invention, the solid state formulation is a powder or cake formulation. Suitable formulations are achieved by spray drying, freeze drying, spray freeze drying and supercritical fluid processing. According to the invention, these methods form a high payload powder or cake solid state formulation that is reconstituted by the hydrogel formulation prior to the transdermal delivery of the VEGF-based agent. Preferably, the powder formulations are adapted to have relatively high porosity to facilitate reconstitution and improve patient compliance.

The noted processes of making powder and cake formulations are highly efficient, typically having yields of approximately 85%. Further, the processes do not require the use of plasticizers that depress Tg and, correspondingly, can reduce shelf life. Preferably, the formulations subjected to drying or supercritical fluid extraction in the noted methods also comprise a carbohydrate, such as a saccharide or a sugar alcohol to help protect the VEGF-based agent. Also preferably, the formulation includes an antioxidant, such as methionine.

Spray drying, freeze drying, spray freeze drying and supercritical fluid extraction afford good control over particle size and distribution, particle shape and morphology. The noted techniques are also known in the art. For example, the spray freeze drying process is ideal for high valued therapeutic drugs as batch sizes as small as 300 mg can be produced with high yields. The noted processes allow production of a solid state formulation that preferably can be reconstituted in up to approximately 15 min, and more preferably in up to approximately 1 min.

As can be appreciated, the spray drying, freeze drying, spray freeze drying and supercritical fluid extraction processes generate a cake form which is readily incorporated into the microprojection system discussed above. Alternatively, the processes generate a powder form, which is further processed to form a cake. In other embodiments, the powder form is held in a container adapted to communicate with the hydrogel. Preferably, such embodiments include stripable release liners to separate the powder form from the hydrogel until reconstitution is desired.

In one embodiment of the invention, a suitable spray freeze drying process generally involves exposing an atomized liquid formulation containing the VEGF-based agent to liquid nitrogen. Under the reduced temperature, the atomized droplets freeze in a time-scale of milliseconds. This freezing process generates very fine ice crystals, which are subsequently lyophilized. The noted technique generates a powder having a high intraparticle porosity, allowing rapid reconstitution in aqueous media

In another embodiment of the invention, a suitable supercritical fluid process generally involves crystallizing a liquid formulation of the VEGF-based agent in a solvent that is maintained above its critical temperature and pressure. Controlling the conditions of the crystallization process allows the production of a VEGF-based agent powder having desired particle size and distribution, particle shape and morphology.

Preferably, the pH of the liquid formulation used to produce the solid state formulation is below about pH 5.5 or above pH 7.0. More preferably, the pH of the formulation used to produce the solid state formulation is in the range of approximately pH 2-pH 5.5 or pH 7.0-pH 11. Even more preferably, the pH of the liquid formulation used to produce the solid state formulation is in the range of approximately pH 2.5-pH 5.5 or pH 7.0-pH 10.5

In another embodiment, the solid state formulation includes a stabilizing agent, which can comprise, without limitation, a non-reducing sugar, a polysaccharide or a reducing sugar.

Suitable non-reducing sugars include, for example, sucrose, trehalose, stachyose, or raffinose.

Suitable polysaccharides include, for example, dextran, soluble starch, dextrin, and insulin.

Suitable reducing sugars include, for example, monosaccharides such as apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as primeverose, vicianose, rutinose, scillabiose, cellobiose, gentiobiose, lactose, lactulose, maltose, melibiose, sophorose, and turanose, and the like.

According to the invention, the solid state formulation can include at least one of the aforementioned pathway patency modulators, vasoconstrictors or buffers.

In accordance with one embodiment of the invention, the method for delivering a VEGF-based agent to a patient includes the following steps: (i) providing a delivery system having a microprojection member, the microprojection member including a plurality of microprojections and a biocompatible coating having at least one VEGF-based agent, (ii) applying the coated microprojection member to the patient's skin, whereby the microprojections pierce the skin and the agent-containing coating is dissolved by body fluid and released into the skin.

The coated microprojection member is preferably left on the patient's skin for a period lasting from 5 seconds to 24 hours. Following the desired wearing time, the microprojection member is removed from the skin.

In accordance with a further embodiment of the invention, the method for delivering a VEGF-based agent to a patient includes the following steps: (i) providing a delivery system having a mnicroprojection member and a gel pack including a hydrogel formulation having at least one VEGF-based agent, (ii) applying the microprojection member-gel pack assembly to the patient's skin, whereby the microprojections pierce the stratum corneum and form a plurality of microslits in the stratum corneum, and whereby the agent-containing hydrogel formulation migrates into and through the microslits formed by the microprojections.

The microprojection member-gel pack assembly is preferably left on the patient's skin for a period lasting from 5 minutes to 24 hours. Following the desired wearing time, the microprojection member-gel pack assembly is removed from the skin.

In a further aspect of the noted embodiment, the microprojection member includes an agent-containing biocompatible coating.

Preferably, the coated microprojection member-gel pack assembly (including the agent-containing hydrogel formulation) is left on the patient's skin for a period lasting from 5 seconds to 24 hours.

In a further aspect of the noted embodiment, the microprojection member includes an agent-containing biocompatible coating and the hydrogel formulation is devoid of a VEGF-based agent and, hence, is merely a hydration mechanism.

Preferably, the coated microprojection member-gel pack assembly (including the agent-containing hydrogel formulation) is left on the patient's skin for a period lasting from 5 minutes to 24 hours.

In accordance with a further embodiment of the invention, the method for delivering a VEGF-based agent to a patient includes the following steps: (i) providing a delivery system having a microprojection member and a gel pack including a hydrogel formulation having at least one VEGF-based agent, (ii) applying the microprojection member to the patient's skin, whereby the microprojections pierce the stratum corneum and form a plurality of microslits in the stratum corneum, and (iii) placing the gel pack on top of the applied microprojection member, whereby the agent-containing hydrogel formulation migrates into and through the microslits formed by the microprojections.

The microprojection member-gel pack assembly is preferably left on the patient's skin for a period lasting from 5 minutes to 24 hours. Following the desired wearing time, the microprojection member-gel pack assembly is removed from the skin.

In a further aspect of the noted embodiment, the microprojection member includes an agent-containing biocompatible coating and the hydrogel formulation is devoid of a VEGF-based agent and, hence, is merely a hydration mechanism.

In accordance with another embodiment of the invention, the method for delivering a VEGF-based agent includes the following steps: (i) providing a delivery system having a microprojection member and a gel pack including a hydrogel formulation having at least one VEGF-based agent, (ii) applying the microprojection member to the patient's skin, whereby the microprojections pierce the stratum corneum and form a plurality of microslits in the stratum corneum, (iii) removing the microprojection member from the patient's skin, and (iv) placing the gel pack on top of the pretreated skin, whereby the agent-containing hydrogel formulation migrates into and through the microslits formed by the microprojections.

The gel pack is preferably left on the patient's skin for a period lasting from 5 minutes to 24 hours. Following the desired wearing time, the gel pack is removed from the skin.

In yet another embodiment of the invention, the microprojection member having a VEGF-based agent containing biocompatible coating is applied to the patient's skin, a gel pack having a VEGF-based agent containing hydrogel formulation is then placed on top of the applied microprojection member, whereby the agent-containing coating is dissolved by body fluid and released into the skin and the agent-containing hydrogel formulation migrates into and through the microslits in the stratum corneum formed by the microprojections.

The microprojection member-gel pack assembly is preferably left on the patient's skin for a period lasting 5 minutes to 24 hours. Following the desired wearing time, the microprojection member and gel pack are removed.

In a further embodiment of the invention, the method for delivering a VEGF-based agent includes the following steps: (i) providing a microprojection assembly having a microprojection member, a hydrogel formulation and a solid state formulation having at least one VEGF-based agent, and (ii) applying the microprojection assembly to the patient's skin, whereby the microprojections pierce the stratum corneum, the hydrogel formulation hydrates and releases the agent formulation from the solid state formulation and the agent migrates into and through the microslits in the stratum corneum formed by the microprojections.

The microprojection member is preferably left on the patient's skin for a period lasting from 5 minutes to 24 hours. Following the desired wearing time, the microprojection member is removed from the skin.

In a further embodiment of the invention, the method for delivering a VEGF-based agent includes the following steps: (i) providing a microprojection assembly having a microprojection member and a solid state formulation having at least one VEGF-based agent, (ii) providing a gel pack having a hydrogel formulation, (iii) applying the microprojection assembly to the patient's skin, whereby the microprojections pierce the stratum corneum, and (iv) placing the gel pack on the applied microprdjection assembly, whereby the hydrogel formulation is released from the gel pack and releases the agent contained in the solid state formulation and the agent and hydrogel formulation migrate into and through the microslits in the stratum corneum formed by the microprojections.

The microprojection member is preferably left on the patient's skin for a period lasting from 5 minutes to 24 hours. Following the desired wearing time, the microprojection member is removed from the skin.

Preferably, the microprojection members and assemblies, and microprojection member-gel pack assemblies employed herein are applied to the patient's skin via an actuator.

Preferably, the dose of VEGF-based agent delivered intracutaneously via the aforementioned methods is in the range of approximately 1-500 μg, more preferably, in the range of approximately 1-100 μg, even more preferably, in the range of approximately 1-200 μg per dosage unit.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by achieved in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field.

Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods, such as sonophoresis or piezoelectric devices, can be used in conjunction with the invention.

### EXAMPLES

The following examples are given to enable those skilled in the art to more clearly understand and practice the present invention. They should not be considered as limiting the scope of the invention but merely as being illustrated as representative thereof.

VEGF Formulation

The following study was performed to develop suitable analytical methods and demonstrate the suitability of VEGF-based agent formulations adapted to coat transdermal delivery microprojection arrays. A 10 mL volume of VEGF (Scios lot# 8331:058 reprocessed #1) was received from Scios. Of this solution, 100 mL (@1.82 mg VEGF/mL) was formulated with 3:1 weight ratio sucrose to VEGF and lyophilized to dryness on a Virtis manifold style freeze drier in four 50 mL centrifuge tubes containing 25 mL liquid formulation each. Following lyophilization, each powder was reconstituted with 2 mL WFI, combined and loaded into a 10,000 MWCO dialysis cassette. The concentrate was dialyzed against 2 mM citrate buffer (pH 4.6) in two 1 L exchanges at 2-8° C. for at least 12 h/exchange. Following dialysis, 21 mL of retentate was recovered from the cassette and analyzed by UV-Vis for protein content, based on a starting concentration of 8.7 mg/mL. The UV content analysis of the incoming raw material and the recovered retentate indicated that 96% of the VEGF peptide was recovered following the first lyophilization and the dialysis processing steps.

The dialysis retentate was then re-formulated with 3:1 weight ratio sucrose:VEGF and lyophilized to dryness on the Virtis freeze drier. Approximately 722 mg of lyophilized powder was recovered and analyzed by HPLC, with the results shown in Table 1.

TABLE 1

| | Purity(%) | | |
|---|---|---|---|
| Sample | RP-HPLC | SEC | IEC |
| 8331:058 raw | 96.6 | 99.6 | 87.0 |
| 2x Lyophilized Powder | 96.4 | 99.2 | 87.7 |
| VEGF Post Coating Solution | 96.2 | 98.0 | 87.5 |
| Coated Array Extract | 96.7 | 99.1 | 88.0 |

Formulation Characteristics

Approximately one-half of the lyophilized powder (332 mg) was reconstituted with 400 μL WFI+0.125% polysorbate 20 to produce ˜750 μL coating solution with a calculated VEGF concentration of 110 mg/mL, 330 mg/mL sucrose and 0.13% polysorbate 20. The powder reconstituted completely with no visible evidence of precipitation or aggregate/fibril formation. A sample of this coating solution was analyzed on a Bohlin C-VOR cone and plate viscometer equipped with a peltier cooler and a 1° cone.

The viscosity for a 70 μL sample was measured as a function of temperature at a shear rate of 400 l/s and also as a function of time at 5° C. and 400 l/s shear rate to determine the stability of the coating solution at constant shear and the presence of any tendency towards gelation under shear for this formulation. The temperature assay indicated that the viscosity of the coating formulation increased slightly as a function of decreasing temperature. This is consistent with previous observations of other peptide coating solutions.

The resulting viscosity of 60 cP at the typical coating temperature of 1° C. is well within the acceptable coatablity parameters for microprojection arrays. The time assay indicated that this coating solution did not have a propensity to gel after two hours under constant shear. A slight increase and decrease in the viscosity was observed that most likely indicates solution concentration and dilution varied over the course of the experiment due to condensation/evaporation of water on the sample stage due to the uncontrolled ambient room humidity.

The viscosity profile as a function of shear rate for this formulation was also assessed at 5° C. The viscosity of the solution was generally insensitive to increases in shear rate and the shear stress of the solution as a function of shear rate fell on a straight line (R2=0.9839), which indicated Newtonian fluid behavior.

The contact angle of the VEGF coating solution was measured on a titanium substrate with a Tantec contact angle meter. As one having skill in the art will recognize, that too great a contact angle, for example, greater than 70°, does not allow good wetting of the titanium tips and renders the formulation “un-coatable.” Similarly, too small a contact angle, for example, less than 30°, renders the solution too wettable. Either very little coating solution remains on the tips after dipping into the solution after each coating pass or the tips wet too deeply along the shaft of the microprojection, leading to undesirable formulation transfer to the base of the array. The contact angle for the 110 mg/mL VEGF solution with 0.13% polysorbate 20 was approximately 54°, a value that is well within the coatable region observed for microprojection arrays coated with other peptide agents.

Coating Feasibility

To demonstrate the coating effectiveness of the formulation, 700 μL of the 110 mg/mL VEGF coating solution was loaded into coating reservoir. A roller coating technique, as described above, was used with a reservoir having Delrin side plates and a 0.621″ stainless steel drum having approximately a 100 μm gap between the doctor blade of the reservoir and the drum. The reservoir was cooled using a peltier cooling stage to a temperature of −1.0° C., resulting in a film temperature on the surface of the drum of between 3 and 4° C.

The drum was rotated at a speed of 50 rpm and strips of microprojection arrays (Macroflux® MF1035 2 cm² arrays, 225 μm nominal tip projection length, available from Alza Corp., Mountain View, Calif.) were coated using a sled height of 250 μm (225 μm tip+25 μm foil backing). The microprojection array strips were coated with 6, 8, 10, 12 and 14 coating passes to generate a coated amount profile and to determine the feasibility of the coating process itself. The coated strips were analyzed with scanning electron micrographs (SEM) and reverse-phase high pressure liquid chromatography (RP-HPLC) for VEGF content using the RP-HPLC content assays described below.

The SEM images indicated the coatings formed by the formulation had a smooth tip-coated morphology that was uniform throughout the microprojection array. Further, the viscosity and the contact angle for this coating formulation were acceptable as the images demonstrated that the coating did not wet too far down the shaft of the microprojection. A RP content assay indicated that the coating amount per coating pass had good linearity in the 6-14 passes region, with approximately 10 g VEGF added at each pass beyond the first 6 coats.

Analytical Method Development

A highly sensitive RP-HPLC method was developed to accommodate low concentration assay for patch residuals and for low concentration skin swab samples to support various in vitro and in vivo preclinical delivery studies. The improved RP protocol employs a small narrow-bore analytical column to enhance the sensitivity of the method as the injected samples are subject to less dilution during separation than with a larger bore column. A mobile phase gradient optimized for the narrow bore column was also employed. The signal-to-noise ratio using this improved method has thus been increased, allowing analyses of VEGF coating amounts equal to or greater than 45 μg of VEGF/array. Conventional assays required greater than 100 μg of VEGF/array. Specifically, using an Agilent Zorbax 2.1×150 mm, C3 and 5 μm narrowbore column, the limit of quantification for RP can be reduced to 0.36 μg for VEGF as compared to the 1.8 μg required with the other methods.

A sample of the coating solution was measured following the one hour coating process for peptide purity by this RP assay and by size-exclusion chromatography (SEC) and ion-exchange chromatography (IEC). These results were compared to a control sample of VEGF, the re-lyophilized powder and extracts of the coated arrays. The data is reproduced at Table 1, as described above. The RP and the IEC assays indicated that the peptide did not show any signs of chemical degradation during the preformulation, coating or final extraction process. The SEC results potentially indicated slightly higher dimer content in the processed material, especially in the post-coating solution. However, it is unclear if the additional aggregation occurred during the coating process, or during sample storage prior to analysis.

Coating Stability

Arrays coated with 80 μg VEGF as described above were assembled with 5 cm² adhesive patches, retainer rings and packaged with a 3.5 g 4 Å molecular sieve desiccant in nitrogen-purged foil pouches and stored under elevated temperature (40° C.) to determine the stability and major degradation pathways. The stressed samples were pulled at T=0, 1 week, 2 weeks and 1 month, and assayed by RP-HPLC, anion-exchange chromatography (AEC) and SEC. The stability data for these systems are summarized in Table 2. The data generated from the stored time points are comparable with samples analyzed at time zero and the drug substance reference standard. These results show good chemical stability of the VEGF-based agent coated microprojection arrays over time, even at the elevated temperature of 40° C.

TABLE 2

| | | |
|---|---|---|
| RP-HPLC | AEC | SEC |

| | | |
|---|---|---|
| Sample | VEGF(μg/array) | Purity (%) |

| | | | |
|---|---|---|---|
| RS 1.82 mg/mL | NA | 87.1 | 99.7 |
| 12-1@T = 0 | 80.2 | 88.0 | 99.1 |
| 1-1 @40 C./1 wk | 82.3 | 87.1 | 99.5 |
| 1-2 @40 C./1 wk | 78.4 | 87.7 | 99.6 |
| 1-3 @40 C./1 wk | 70.9 | 87.2 | 99.6 |
| Mean | 77.2 | 87.3 | 99.6 |
| 2-1 @40 C./2 wk | 78.0 | 87.5 | 99.5 |
| 2-2 @40 C./2 wk | 76.0 | 87.3 | 99.5 |
| 2-3 @40 C./2 wk | 76.1 | 87.5 | 99.5 |
| Mean | 76.7 | 87.4 | 99.5 |
| 3-1 @40 C./1 M | 96.9 | 86.8 | 99.3 |
| 3-2 @40 C./1 M | 97.8 | 86.2 | 99.3 |
| 3-3 @40 C./1 M | 97.8 | 86.4 | 99.3 |
| Mean | 97.5 | 86.5 | 99.3 |

VEGF Delivery with Coated Arrays

Different coated array patches were applied to hairless guinea pigs (HGP) to test the feasibility of drug delivery. Specifically, a preclinical study was conducted to evaluate the tolerability efficiency of drug delivery for microprojection arrays coated as described above with 24 kg, 42 kg and 80 μg of VEGF using the 0.29J force applicator. A residual assay was performed on arrays that had been applied to HGP and removed in order to determine the quantity of drug remaining on the array. These residual amounts are compared to control in Table 3, thus indicating delivery amounts. Together with related erythema/edema scores, this study indicated that most of the coated VEGF was delivered from the microprojection into the HGP skin without significant local reaction.

TABLE 3

| | | Residual | | Control |
|---|---|---|---|---|
| | Residual | Mean | Control | Mean |
| Sample ID | (μg/array) | (μg/array) | (μg/array) | (μg/array) |
| #1, 24 μg, applied | BQL | 0.9 | 27.1 | 30.7 |
| | (<1.82 μg/array) | | | |
| | 2.8 | | 29.1 | |
| | 0.0 | | 36.0 | |
| #2, 42 μg, applied | 2.8 | 4.5 | 46.2 | 45.8 |
| | 3.7 | | 46.4 | |
| | 7.0 | | 44.7 | |
| #3, 80 μg, applied | 10.3 | 9.7 | 93.1 | 94.1 |
| | 10.4 | | 97.1 | |
| | 8.4 | | 92.0 | |

In summary, the above examples show that a VEGF-based agent concentrated to 10 mg/mL, dialyzed to reduce citrate content, lyophilized a 3:1 sucrose:VEGF formulation and reconstituted with 0.125% polysorbate 20 can produce 0.75 mL coating solution with acceptable viscosity and contact angle for the microprojection tip coating process. The formulation can be employed to successfully coat microprojection arrays with VEGF in amounts in the range of approximately 20 kg to 90 μg VEGF. Testing demonstrated that the coated VEGF had similar purity and RP-HPLC profile to the original API solution. Additional HPLC testing (IC, SEC) determined that the purity of the API remains intact during formulation, coating and storage at elevated temperatures. Further, it was determined that coated arrays had good linearity in the range of approximately 5 to 200 kg VEGF/mL.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A device for transdermally delivering a VEGF-based agent to a patient, comprising:
a microprojection member having a plurality of microprojections that are adapted to pierce the stratum corneum of the patient; and
a biocompatible coating disposed on said microprojection member, said coating being formed from a coating formulation having at least one VEGF-based agent disposed thereon.

2. The device of claim 1, wherein said coating is disposed on at least one of said plurality of microprojections.

3. The device of claim 1, wherein said coating formulation comprises an aqueous formulation.

4. The device of claim 1, wherein said coating formulation comprises a non-aqueous formulation.

5. The device of claim 1, wherein said VEGF-based agent is selected from the group consisting of isoforms of VEGF 206, VEGF 189, VEGF 183, VEGF 165, VEGF 148, VEGF 145 and VEGF 121, and salts and simple derivatives thereof.

6. The device of claim 5, wherein said VEGF 121 salt is selected from the group consisting of acetate, propionate, butyrate, pentanoate, hexanoate, heptanoate, levulinate, chloride, bromide, citrate, succinate, maleate, glycolate, gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate and sulfonate.

7. The device of claim 1, wherein said VEGF-based agent comprises in the range of approximately 1-30 wt. % of said coating formulation.

8. The device of claim 1, wherein said VEGF-based agent comprises in the range of 1 μg -1000 μg of said biocompatible coating.

9. The device of claim 1, wherein the pH of said coating formulation is below approximately pH 5.5.

10. The device of claim 1, wherein the pH of said coating formulation is above approximately pH 7.0

11. The device of claim 1, wherein said coating formulation includes at least one low volatility counterion.

12. The device of claim 1, wherein said coating formulation includes a plurality of counterions.

13. The device of claim 9, wherein said VEGF-based agent has a positive charge at said coating formulation pH and wherein said coating formulation includes at least one acidic counterion.

14. The device of claim 12, wherein at least one of said plurality of counterions comprises a first weak acid and at least one of said plurality of counterions comprise a second non-volatile weak acid.

15. The device of claim 12, wherein at least one of said plurality of counterions comprises a second weak acid having a high volatility.

16. The device of claim 10, wherein said VEGF-based agent has a negative charge at said coating formulation pH and wherein said coating formulation includes at least a second counterion comprising a base.

17. The device of claim 12, wherein at least one of said plurality of counterions comprises a first strong base and at least one of said plurality of counterions comprises a second weak base.

18. The device of claim 12, wherein at least one of said plurality of counterions comprises a second strong base and at least one of said plurality of counterions comprises a third weak base.

19. The device of claim 11, wherein the amount of said low volatility counterion present in said coating formulation is sufficient to neutralize the charge of said VEGF-based agent.

20. The device of claim 1, wherein said VEGF-based agent comprises VEGF 121 and wherein said coating formulation includes at least one viscosity-enhancing counterion.

21. The device of claim 1, wherein said coating formulation has a viscosity in the range of approximately 3-500 centipose.

22. The device of claim 1, wherein the thickness of said biocompatible coating is less than approximately 25 microns.

23. A delivery system for transdermally delivering a VEGF-based agent to a patient, comprising:
a microprojection member having a plurality of microprojections that are adapted to pierce the stratum corneum of the patient; and
a hydrogel formulation having at least one VEGF-based agent, said hydrogel formulation being in communication with said microprojection member.

24. The delivery system of claim 23, wherein said VEGF-based agent comprises in the range of approximately 1-40 wt. % of said hydrogel formulation.

25. The delivery system of claim 23, wherein said VEGF-based agent is selected from the group consisting of isoforms of VEGF 206, VEGF 189, VEGF 183, VEGF 165, VEGF 148, VEGF 145 and VEGF 121, and salts and simple derivatives thereof.

26. The delivery system of claim 23, wherein said hydrogel formulation comprises a water-based hydrogel having a macromolecular polymeric network.

27. The delivery system of claim 23, wherein said hydrogel formulation includes at least one surfactant, selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, sorbitan derivatives, and alkoxylated alcohols.

28. A delivery system for transdermally delivering a VEGF-based agent to a patient, comprising:
a microprojection member having a plurality of microprojections that are adapted to pierce the stratum corneum of the patient;
a solid state formulation disposed proximate said microprojection member; and
a hydrogel formulation, said hydrogel formulation being in communication with said solid state formulation.

29. The delivery system of claim 28, wherein said VEGF-based agent is selected from the group consisting of isoforms of VEGF 206, VEGF 189, VEGF 183, VEGF 165, VEGF 148, VEGF 145 and VEGF 121, and salts and simple derivatives thereof.

30. The delivery system of claim 28, wherein said solid state formulation is a solid film is made by casting a liquid formulation comprising at least one VEGF-based agent, a polymeric material, a plasticizing agent, a surfactant and a volatile solvent.

31. The delivery system of claim 28, wherein said solid state formulation is formed by a process selected from the group consisting of spray drying, freeze drying, spray freeze drying and supercritical fluid extraction.

32. A method of transdermally delivering a VEGF-based agent to a patient, comprising the steps of:
providing a microprojection member having a plurality of microprojections, said microprojection member having a coating disposed thereon, said coating including at least one VEGF-based agent;
applying said microprojection member to a skin site of said patient, whereby said plurality of microprojections pierce the stratum corneum and deliver said VEGF-based agent to said patient; and
removing said microprojection member from said skin site.

33. The method of claim 32, wherein said microprojection member remains applied to said skin site for a period of time in the range of 5 sec. to 24 hrs.

34. The method of claim 32, wherein said VEGF-based agent comprises in the range of approximately 1 μg-1000 μg of said biocompatible coating.

35. A method for transdermally delivering a VEGF-based agent to a patient, comprising the steps of:
providing a microprojection assembly having a microprojection member and a gel pack, said microprojection member including a plurality of microprojections, said gel pack including a hydrogel formulation having at least one VEGF-based agent;
applying said microprojection member-gel pack assembly to a skin site of said patient, whereby a plurality of microslits are formed in the patient's stratum-corneum, and whereby said hydrogel formulation is released from said gel pack and migrates into and through said microslits formed by said microprojections; and
removing said microprojection member-gel pack assembly from said skin site.

36. The method of claim 35, wherein said microprojection member-gel pack assembly remains applied to said skin site for a period of time in the range of 5 min. to 24 hrs.

37. The method of claim 35, wherein said microprojection member includes a biocompatible coating having at least one VEGF-based agent.

38. The method of claim 35, wherein said microprojection-gel pack assembly remains applied to said skin site for a period of time in the range of 5 sec. to 24 hours.

39. The method of claim 35, wherein VEGF-based agent is selected from the group consisting of isoforms of VEGF 206, VEGF 189, VEGF 183, VEGF 165, VEGF 148, VEGF 145 and VEGF 121, and salts and simple derivatives thereof.

40. The method of claim 35, wherein said hydrogel formulation is devoid of a VEGF-based agent.

41. A method for transdermally delivering a VEGF-based agent to a patient, comprising the steps of:
providing a microprojection assembly having a microprojection member and a gel pack, said microprojection member including a plurality of microprojections, said gel pack including a hydrogel formulation having at least one VEGF-based agent;
applying said microprojection member to a skin site of said patient, whereby a plurality of microslits are formed in the patient's stratum-corneum;
placing said gel pack on said microprojection member, whereby said hydrogel formulation is released from said gel pack and migrates into and through said microslits formed by said microprojections; and
removing said microprojection member from said skin site.

42. The method of claim 41, wherein said gel pack includes a release liner and said method includes the step of removing said release liner prior to placing said gel pack on said microprojection member.

43. The method of claim 41, wherein said microprojection member-gel pack assembly remains applied to said skin site for a period of time in the range of 5 min. to 24 hrs.

44. The method of claim 41, wherein said microprojection member includes a biocompatible coating having at least one VEGF-based agent.

45. The method of claim 41, wherein VEGF-based agent is selected from the group consisting of isoforms of VEGF 206, VEGF 189, VEGF 183, VEGF 165, VEGF 148, VEGF 145 and VEGF 121, and salts and simple derivatives thereof.

46. The method of claim 41, wherein said VEGF-based agent comprises in the range of approximately 0.1-2 wt. % of said hydrogel formulation.

47. A method for transdermally delivering a VEGF-based agent to a patient, comprising the steps of:
providing a microprojection assembly having a microprojection member and a gel pack, said microprojection member including a plurality of microprojections, said gel pack including a hydrogel formulation having at least one VEGF-based agent;
applying said microprojection member to a skin site of said patient, whereby a plurality of microslits are formed in the patient's stratum-corneum;
removing said microprojection member from said skin site;
placing said gel pack on said pre-treated skin site, whereby said hydrogel formulation is released from said gel pack and migrates into and through said microslits formed by said microprojections; and
removing said gel pack from said skin site.

48. The method of claim 47, wherein said gel pack remains applied to said pre-treated skin site for a period of time in the range of 5 min. to 24 hrs.

49. The method of claim 47, wherein said VEGF-based agent is selected from the group consisting of isoforms of VEGF 206, VEGF 189, VEGF 183, VEGF 165, VEGF 148, VEGF 145 and VEGF 121, and salts and simple derivatives thereof.

50. A method for transdermally delivering a VEGF-based agent to a patient, comprising the steps of:
providing a microprojection assembly having a microprojection member, a gel pack and a solid state formulation, said microprojection member including a plurality of microprojections, said gel pack including a hydrogel formulation, said solid state formulation being disposed proximate said microprojection member and including at least one VEGF-based agent;
applying said microprojection assembly to a skin site of said patient, whereby a plurality of microslits are formed in the patient's stratum-corneum, and whereby said hydrogel formulation is released from said gel pack and releases said agent contained in said solid state formulation and said agent and hydrogel formulation migrates into through said microslits formed by said microprojections; and
removing said microprojection assembly from said skin site.

51. The method of claim 50, wherein said microprojection assembly remains applied to said skin site for a period of time in the range of 5 min. to 24 hrs.

52. The method of claim 50, wherein said VEGF-based agent is selected from the group consisting of isoforms of VEGF 206, VEGF 189, VEGF 183, VEGF 165, VEGF 148, VEGF 145 and VEGF 121, and salts and simple derivatives thereof.

53. A method for transdermally delivering a VEGF-based agent to a patient, comprising the steps of:
providing a microprojection assembly having a microprojection member and a gel pack, said microprojection member including a plurality of microprojections, said solid state formulation being disposed proximate said microprojection member and including at least one VEGF-based agent;
providing a gel pack having a hydrogel formulation;
applying said microprojection assembly to a skin site of said patient, whereby a plurality of microslits are formed in the patient's stratum-corneum;
placing said gel pack on said microprojection assembly, whereby said hydrogel formulation is released from said gel pack and releases said agent contained in said solid state formulation and said agent and hydrogel formulation migrates into through said microslits formed by said microprojections; and
removing said microprojection assembly from said skin site.

54. The method of claim 53, wherein said gel pack includes a release liner and said method includes the step of removing said release liner prior to placing said gel pack on said microprojection assembly.

55. The method of claim 53, wherein said microprojection assembly-gel pack remains applied to said skin site for a period of time in the range of 5 min. to 24 hrs.

56. The method of claim 53, wherein said VEGF-based agent is selected from the group consisting of isoforms of VEGF 206, VEGF 189, VEGF 183, VEGF 165, VEGF 148, VEGF 145 and VEGF 121, and salts and simple derivatives thereof.
